(19) United States
(12) Patent Application Publication
(10) Pub. No.: US 2010/0150914 A1
(43) Pub. Date: Jun. 17, 2010

(54) AGONIST TRKB ANTIBODIES AND USES THEREOF

(75) Inventors: Yan Wang, South San Francisco, CA (US); Steven B. Cohen, San Diego, CA (US); Marc Nasoff, Rancho Santa Fe, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/516,187

(22) PCT Filed: Nov. 6, 2007
(86) PCT No.: PCT/US2007/083774

§ 371 Date: May 22, 2009

Related U.S. Application Data
(60) Provisional application No. 60/858,169, filed on Nov. 9, 2006.

(51) Int. Cl. A61K 39/395; C07K 16/18; C07K 16/28; A61P 3/10

(52) U.S. Cl. 424/133.1; 530/387.1; 530/387.3; 424/172.1

(57) ABSTRACT

TrkB agonist antibodies and methods of their use are provided.

### CROSS-REFERENCE TO RELATED PATENT APPLICATION

The present application claims benefit to U.S. Provisional Patent Application 60/858,169, filed Nov. 9, 2006, which is incorporated by reference in its entirety for all purposes.

### BACKGROUND OF THE INVENTION

I. TrkB

Tyrosine receptor kinase B (TrkB) belongs to a family of single transmembrane receptor tyrosine kinases that includes TrkA and TrkC. These tyrosine receptor kinases (trks) mediate the activity of neurotrophins Neurotrophins are required for neuronal survival and development and regulate synaptic transmission via modulation of neuronal architecture and synaptic plasticity. Neurotrophins include, but are not limited to, nerve growth factor (NGF), brain derived neurotrophic factor (BDNF), neurotrophin-3 (NT-3), and neurotrophin-4/5 (NT-4/5). (Lo, K Y et al., J. Biol. Chem., 280:41744-52 (2005)). TrkB is a high affinity receptor of BDNF (Minichiello, et al., Neuron 21:335-45 (1998)). Neurotrophin binding to trk activates the receptor which dimerizes and auto-phosphorylates specific tyrosine residues on the intracellular domain of the receptor (Jing, et al. Neuron 9:1067-1079 (1992); Barbacid, J. Neurobiol. 25:1386-1403 (1994); Bothwell, Ann. Rev. Neurosci. 18:223 253 (1995); Segal and Greenberg, Ann. Rev. Neurosci. 19:463 489 (1996); Kaplan and Miller, Curr. Opinion Neurobiol. 10:381 391 (2000)). These phospho-tyrosine residues serve as docking sites for elements of intracellular signaling cascades that lead to the suppression of neuron death and other effects of the neurotrophins For example, Shc, FRS-2, SH2B, rAPS and PLCγ interact with TrkB via phosphorylated tyrosine residues. Association of these adaptor molecules with activated TrkB results in the initiation of signaling pathways, including the mitogen-activated protein kinase, phosphatidylinositol 3-kinase, and PLCγ pathways, thereby mediating the actions of neurotrophins (Lo, K Y et al., J. Biol. Chem., 280:41744-52 (2005)).

II. Diabetes

The concentration of glucose in the human bloodstream must be controlled within a relatively tight range (60-120 milligrams per deciliter of blood) to maintain normal health. If blood glucose drops too low, a condition known as hypoglycemia results, with symptoms such as faintness, weakness, headache, confusion and personality changes. Excessive blood glucose, or hyperglycemia, may cause tissue damage due to the chemical reactions between the excess glucose and proteins in cells, tissues, and organs. This damage is thought to cause the diabetic complications of blindness, kidney failure, impotence, atherosclerosis, and increased vulnerability to infection.

Diabetes mellitus is associated with continuous and pathologically elevated blood glucose concentration; it is one of the leading causes of death in the United States and is responsible for about 5% of all mortality. Diabetes is divided into two major sub-classes: Type I, also known as juvenile diabetes, or Insulin-Dependent Diabetes Mellitus (IDDM), and Type II, also known as adult onset diabetes, or Non-Insulin-Dependent Diabetes Mellitus (NIDDM).

The diagnosis of Type II diabetes mellitus includes assessment of symptoms and measurement of glucose in the urine and blood. Blood glucose level determination is necessary for an accurate diagnosis. More specifically, fasting blood glucose level determination is a standard approach used. However, the oral glucose tolerance test (OGTT) is considered to be more sensitive than fasted blood glucose level. Type II diabetes mellitus is associated with impaired oral glucose tolerance (OGT). The OGTT thus can aid in the diagnosis of Type II diabetes mellitus, although generally not necessary for the diagnosis of diabetes (Emancipator K, Am J Clin Pathol 1997 November; 112(5):665 74; Type 2 Diabetes Mellitus, Decision Resources Inc., March 2000).

Thus, impaired glucose tolerance is diagnosed in individuals that have fasting blood glucose levels less than those required for a diagnosis of Type II diabetes mellitus, but have a plasma glucose response during the OGTT between normal and diabetics Impaired glucose tolerance is considered a prediabetic condition, and impaired glucose tolerance (as defined by the OGTT) is a strong predictor for the development of Type II diabetes mellitus (Haffner S M, Diabet Med 1997 August; 14 Suppl 3:S12 8).

Type II diabetes mellitus is a progressive disease associated with the reduction of pancreatic function and/or other insulin-related processes, aggravated by increased plasma glucose levels. Thus, Type II diabetes mellitus usually has a prolonged prediabetic phase and various pathophysiological mechanisms can lead to pathological hyperglycemia and impaired glucose tolerance, for instance, abnormalities in glucose utilization and effectiveness, insulin action and/or insulin production in the prediabetic state (Goldberg R B, Med Clin North Am 1998 July; 82(4):805 21).

The prediabetic state associated with glucose intolerance can also be associated with a predisposition to abdominal obesity, insulin resistance, hyperlipidemia, and high blood pressure (Groop L, Forsblom C, Lehtovirta M, Am J Hypertens 1997 September; 10(9 Pt 2):1725 180S; Haffner S M, J Diabetes Complications 1997 March-April, 11(2):69 76; Beck-Nielsen H, Henriksen J E, Alford F, Hother-Nielson O, Diabet Med 1996 September; 13 (9 Suppl 6):578 84).

Early intervention in individuals at risk to develop Type II diabetes mellitus, focusing on reducing the pathological hyperglycemia or impaired glucose tolerance may prevent or delay the progression towards Type II diabetes mellitus and associated complications. Therefore, by effectively treating impaired oral glucose tolerance and/or elevated blood glucose levels, one can prevent or inhibit the progression of the disorder to Type II diabetes mellitus. See, e.g., U.S. Pat. No. 7,109,174.

Insulin and sulfonylureas (oral hypoglycemia therapeutic agents) are the two major classes of diabetes medicines prescribed today in the United States. Insulin is prescribed for both Type I and Type II diabetes, while sulfonylureas are usually prescribed for Type II diabetics only. Sulfonylureas stimulate natural insulin secretion and reduce insulin resistance; these compounds do not replace the function of insulin in metabolism. Approximately one-third of patients who receive sulfonylurea become resistant to it. Some Type II diabetics do not respond to sulonylurea therapy. Of patients who do respond to initial treatment with sulfonylureas, 5-10% are likely to experience a loss of sulfonylurea effectiveness after about ten years. See, e.g., U.S. Pat. No. 7,115,284.

Many anti-diabetic agents typically prescribed for the treatment of Type II diabetes mellitus, for example, sulfonylureas and thiazolidinediones, have an undesired side effect of increasing body weight. Increased body weight in patients with prediabetic conditions or with diagnosed Type II diabetes mellitus results in deleterious effects due to accentuation of the metabolic and endocrine dysregulation, and obesity per se is a pivotal risk factor for the development and progressive worsening of Type II diabetes mellitus. Thus it is desirable to have an anti-diabetic agent which maintains or lowers body weight. See, e.g., U.S. Pat. No. 7,199,174.

Obesity is a common and very serious public health problem as it increases a person's risk for a number of serious conditions, including diabetes, heart disease, stroke, high blood pressure, and some types of cancers. Considerable increase in the number of obese individuals over the past two decades has created profound public health implications. Although studies have demonstrated that reduction in obesity by diet and exercise reduces the associated risk factors dramatically, these treatments are largely unsuccessful considering obesity is strongly associated with genetically inherited factors that contribute to increased appetite, preferences for highly caloric foods, reduced physical activity, and increased lipogenic metabolism. See, e.g., U.S. Pat. No. 7,115,767. Thus, it is an object of the invention to address the shortcomings in current hyperglycemia, obesity, and diabetes treatments.

### BRIEF SUMMARY OF THE INVENTION

The present invention provides isolated antibody agonists of Tyrosine Kinase Receptor B (TrkB). In some embodiments, the antibody is a humanized antibody. In some embodiments, the antibody is a single chain antibody. In some embodiments, the antibody does not bind to Tyrosine Kinase Receptor A or Tyrosine Kinase Receptor C.

In some embodiments, the antibody binds to the Ligand Binding Domain (LBD) of TrkB. In some embodiments, the antibody competes with the binding of Brain Derived Neurotrophic Factor (BDNF) to TrkB. In some embodiments, the antibody competes for binding to TrkB with a competitor antibody comprising a heavy chain variable region comprising SEQ ID NO:3 and a light chain variable region comprising SEQ ID NO:4. In some embodiments, the antibody comprises a heavy chain variable region comprising SEQ ID NO:7 and a light chain variable region comprising SEQ ID NO:8. In some embodiments, the antibody comprises a heavy chain variable region comprising SEQ ID NO:11 and a light chain variable region comprising SEQ ID NO:12. In some embodiments, the antibody comprises a heavy chain variable region comprising SEQ ID NO:15 and a light chain variable region comprising SEQ ID NO:16. In some embodiments, the antibody comprises a heavy chain variable region comprising SEQ ID NOs:7, 11, and 15 and a light chain variable region comprising SEQ ID NOs:8, 12, and 16. In some embodiments, the antibody comprises a heavy chain variable region comprising SEQ ID NO:3 and a light chain variable region comprising SEQ ID NO:4.

In some embodiments, the antibody does not bind to the ligand binding domain of TrkB. In some embodiments, the antibody does not compete with the binding of Brain Derived Neurotrophic Factor (BDNF) to TrkB. In some embodiments, the antibody competes for binding to TrkB with a competitor antibody comprising a heavy chain variable region comprising SEQ ID NO:1 and a light chain variable region comprising SEQ ID NO:2. In some embodiments, the antibody comprises a heavy chain variable region comprising SEQ ID NO:5 and a light chain variable region comprising SEQ ID NO:6. In some embodiments, the antibody comprises a heavy chain variable region comprising SEQ ID NO:9 and a light chain variable region comprising SEQ ID NO:10. In some embodiments, the antibody comprises a heavy chain variable region comprising SEQ ID NO:13 and a light chain variable region comprising SEQ ID NO:14. In some embodiments, the antibody comprises a heavy chain variable region comprising SEQ ID NOs:5, 9, and 13 and a light chain variable region comprising SEQ ID NOs:6, 10, and 14. In some embodiments, the antibody comprises a heavy chain variable region comprising SEQ ID NO:1 and a light chain variable region comprising SEQ ID NO:2.

The present invention also provides physiological compositions comprising, a therapeutically effective amount of the antibody of claim 1; and a pharmaceutical carrier. In some embodiments, the pharmaceutical composition further comprises an agent that reduces blood glucose levels in an individual. In some embodiments, the pharmaceutical composition further comprises an agent that reduces body weight in an individual.

The present invention also provides methods of reducing blood glucose levels and/or body weight in an individual in need thereof. In some embodiments, the methods comprise administering a therapeutically effective amount of an antibody agonist of Tyrosine Kinase Receptor B (TrkB) to the individual. In some embodiments, the individual is pre-diabetic. In some embodiments, the individual has type I diabetes. In some embodiments, the individual has type II diabetes. In some embodiments, the individual is overweight. In some embodiments, the individual is obese.

In some embodiments, a therapeutically effective amount of a second agent effective in reducing blood glucose is administered to the individual in combination with the antibody agonist of TrkB. In some embodiments, the second agent and the antibody agonist of TrkB are administered as a mixture. In some embodiments, the second agent is administered separately from the antibody agonist of TrkB. In some embodiments, the second agent is selected from the group consisting of: insulin, sulfonylureas, insulinotropic agents, metformin, PPARγ agonists PPARα agonists, PPARδ agonists, PPARα/γ dual agonists, PPARα/γ/δ pan agonists, alpha-glucosidase inhibitors, DPP-IV inhibitors, and GLP-1/GLP-1 analogs.

In some embodiments, a therapeutically effective amount of a second agent effective in reducing weight or obesity is administered to the individual in combination with the antibody agonist of TrkB. In some embodiments, the second agent and the antibody agonist of TrkB are administered as a mixture. In some embodiments, the second agent is administered separately from the antibody agonist of TrkB. In some embodiments, the second agent is selected from the group consisting of lipase inhibitors, sibutramine, CB-1 inhibitors, topiramate, amylin, amylin analogs, leptin, PYY/PYY analogs, and GLP-1/GLP-1 analogs.

### DEFINITIONS

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by, e.g., Kabat et al. (Kabat et al., Sequences of Proteins of Immunological Interest, Fifth Edition, U.S. Department of Health and Human Services, U.S. Government Printing Office (1991)).

An exemplary naturally-occurring immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see FUNDAMENTAL IMMUNOLOGY (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term “antibody”, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. This selection may be achieved by subtracting out antibodies that cross-react with, e.g., TrkA or TrkC. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The term “antibody agonist” refers to an antibody capable of activating a receptor to induce a full or partial receptor-mediated response. For example, an agonist of TrkB binds to TrkB and induces TrkB-mediated signaling. In some embodiments, a TrkB antibody agonist can be identified by its ability to bind TrkB and induce neurite outgrowth when contacted to SH-SY5Y cells or as otherwise described herein. Agonist antibodies are those that activate a receptor response at least 10% above the response in the absence of the antibody. In some cases, an agonist antibody activates a receptor response more than 25%, 50%, 75%, or 100% above the response in the absence of the antibody. Some antibody agonists activate a receptor response of 200%, 300%, 400%, 500%, or more above the response in the absence of the antibody.

“Activity” of a polypeptide of the invention refers to structural, regulatory, or biochemical functions of a polypeptide in its native cell or tissue. Examples of activity of a polypeptide include both direct activities and indirect activities. Exemplary direct activities are the result of direct interaction with the polypeptide, including ligand binding, such as binding of BDNF to the Ligand Binding Domain (LBD) (see, e.g., Naylor et al., Biochem Biophys Res Commun. 291(3):501-7 (2002) and SEQ ID NO:18) of TrkB, production or depletion of second messengers (e.g., cAMP, cGMP, IP₃, DAG, or Ca²⁺), ion flux, and changes in the level of phosphorylation or transcription. Exemplary indirect activities in the context of TrkB are observed as a change in phenotype or response in a cell or tissue to a polypeptide's directed activity, e.g., reducing overall blood glucose levels as a result of the interaction of the polypeptide with other cellular or tissue components.

The term “obese,” when used in reference to adult humans, refers to an individual with a body mass index (BMI) of 30 or more. “Overweight,” when used in reference to adult humans, refers to an individual with a BMI of 25 or more. For children, the charts of Body-Mass-Index for Age are used, where a BMI greater than the 85^{th} percentile is considered “overweight” and a BMI greater than the 95^{th} percentile is considered “obese”. See, e.g., Clinical Guidelines on the Identification, Evaluation, and Treatment of Overweight and Obesity in Adult (National Heart, Lung and Blood Institute, Jun. 17, 1998) and Preventing and Managing the Global Epidemic of Obesity in Report of the World Health Organization Consultation of Obesity (WHO, Geneva, June 1997).

A “pre-diabetic individual” refers to an adult with a fasting blood glucose level greater than 110 mg/dl but less than 126 mg/dl or a 2 hour PG reading of greater than 140 mg/dl but less than 200 mg/dl. A “diabetic individual,” when used to compare with a sample from a patient, refers to an adult with a fasting blood glucose level greater than 126 mg/dl or a 2 hour PG reading of greater than 200 mg/dl. “Fasting” refers to no caloric intake for at least 8 hours. A “2 hour PG” refers to the level of blood glucose after challenging a patient to a glucose load containing the equivalent of 75 g anhydrous glucose dissolved in water. The overall test is generally referred to as an oral glucose tolerance test (OGTT). See, e.g., Diabetes Care, 2003, 26(11): 3160-3167 (2003).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The terms “nucleic acid” and “polynucleotide” are used interchangeably.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, designated region as measured using one of the following sequence comparison algorithms or by manual alignment and visual inspection, or across the entire sequence where not indicated. The invention provides polypeptides or polynucleotides encoding polypeptides that are substantially identical, or comprising sequences substantially identical, to the polypeptides exemplified herein (e.g., SEQ ID NO: 1, 2, 3, 4, 5, 6, 7, 8, 9, 10, 11, 12, 13, 14, 15, 16, 17, 18). This definition also refers to the complement of a nucleotide test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### BRIEF DESCRIPTION OF THE DRAWINGS

FIG. 1 illustrates effect of BDNF in an Anoikis assay.

FIG. 2 shows the effect of various identified TrkB antibodies in an Anoikis assay.

FIG. 3 displays thereactivity of the isolated TrkB antibodies.

FIG. 4 shows that the purified TrkB functional antibody agonists are not reactive against human TrkA or TrkC.

FIG. 5 shows antibody agonists in a SH-SY5Y differentiation assay.

FIG. 6 shows isotyping results of TrkB monoclonal functional antibodies.

FIG. 7 summarizes information for various agonist antibodies identified.

FIG. 8 illustrates that TrkB agonist antibodies reduce serum glucose levels and promote weight loss prophylactically.

FIG. 9 illustrates that TrkB agonist antibodies reduce serum glucose levels and promote weight loss prophylactically and therapeutically.

FIG. 10 provides the variable region sequences of the A10 and C20 antibodies disclosed herein. For each sequence, the first underlined portion is CR1, the second underlined portion is CDR2 and the third underlined portion is CDR3.

### DETAILED DESCRIPTION OF THE INVENTION

### I. Introduction

The present invention provides novel TrkB agonist antibodies. The TrkB agonist antibodies of the invention bind specifically to TrkB and activate TrkB. Surprisingly, the present application demonstrates that the TrkB agonist antibodies of the invention also dramatically reduce blood glucose levels in diabetic mice in vivo. Further, treatment with the TrkB agonist antibodies of the invention prevented the weight gain that is normally observed in these mice. These results demonstrate that the antibodies of the invention are specific for TrkB and are effective in activating the receptor. Moreover, the results reveal that TrkB activation is useful in preventing hyperglycemia and its associated conditions, obesity, pre-diabetes, and type-II diabetes.

### II. Antibodies that Bind TrkB

1. Introduction

Any TrkB agonist antibodies can be used according to the methods of the invention.

In some embodiments, TrkB agonist antibodies of the invention bind to the TrkB ligand binding site and/or competes with BDNF for binding to TrkB. An exemplary antibody that binds to the ligand binding site of TrkB is Antibody A10F18.2 (also referred to herein as “A10F18” or “A10”). The heavy chain variable region of antibody A10 is exemplified in SEQ ID NO:1 and the light chain variable region of antibody A10 is exemplified in SEQ ID NO:2. Accordingly, the invention provides agonist antibodies that compete for binding to TrkB with an antibody comprising a heavy chain variable region comprising SEQ ID NO:1 and a light chain variable region comprising SEQ ID NO:2. In some embodiments, the antibodies of the invention comprise at least of the complementarity determining regions (CDRs) of SEQ ID NO:1 and/or 2. Without intending to limit the scope of the invention, it is believed that CDR3 plays a significant role in the binding of antibody A10. Accordingly, in some embodiments, an antibody of the present invention comprises SEQ ID NOs: 5 and/or 6. However, CDR1 and/or CDR2 also play a role in binding. Accordingly, in some embodiments, an antibody of the present invention comprises SEQ ID NOs: 9 and/or 10 or 13 and/or 14.

In some embodiments, TrkB agonist antibodies of the invention do not bind to the TrkB ligand binding site and/or compete with BDNF for binding to TrkB. An exemplary antibody that does not bind to the ligand binding site of TrkB is Antibody C20.i1.1 (also referred to herein as “C20.i1”, “C20.i1” and “C20”). The heavy chain variable region of antibody C20 is exemplified in SEQ ID NO:3 and the light chain variable region of antibody C20 is exemplified in SEQ ID NO:4. Accordingly, the invention provides agonist antibodies that compete for binding to TrkB with an antibody comprising a heavy chain variable region comprising SEQ ID NO:3 and a light chain variable region comprising SEQ ID NO:4. In some embodiments, the antibodies of the invention comprise at least of the complementarity determining regions (CDRs) of SEQ ID NO:3 and/or 4. Without intending to limit the scope of the invention, it is believed that CDR3 plays a significant role in the binding of antibody C20. Accordingly, in some embodiments, an antibody of the present invention comprises SEQ ID NOs: 7 and/or 8. However, CDR1 and/or CDR2 also play a role in binding. Accordingly, in some embodiments, an antibody of the present invention comprises SEQ ID NOs: 11 and/or 12 or 15 and/or 16.

Any type of antibody agonist may be used according to the methods of the invention. Generally, the antibodies used are monoclonal antibodies. Monoclonal antibodies can be generated by any method known in the art (e.g., using hybridomas, recombinant expression and/or phage display).

2. Humanized Antibodies

In some embodiments, the antibody used according to the present invention is a chimeric (e.g., mouse/human) antibody made up of regions from an non-human anti-TrkB antibody agonist together with regions of human antibodies. For example, a chimeric H chain can comprise the antigen binding region of the heavy chain variable region (e.g., SEQ ID NOs:1 or 3 or at least parts thereof, such as a CDR) of the non-human antibody linked to at least a portion of a human heavy chain constant region. This humanized or chimeric heavy chain may be combined with a chimeric L chain that comprises the antigen binding region of the light chain variable region (e.g., SEQ ID NOs:2 or 4 or at least parts thereof, such as a CDR) of the non-human antibody linked to at least a portion of the human light chain constant region. In some embodiments, the heavy chain constant region can be an IgM or IgA antibody.

The chimeric antibodies of the invention may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

The DNA sequences of the antibodies of the invention can be identified, isolated, cloned, and transferred to a prokaryotic or eukaryotic cell for expression by procedures well-known in the art. Such procedures are generally described in Sambrook et al., supra, as well as CURRENT PROTOCOLS IN MOLECULAR BIOLOGY (Ausubel et al., eds., 1989). Expression vectors and host cells suitable for expression of recombinant antibodies and humanized antibodies in particular, are well known in the art. The following references are representative of methods and vectors suitable for expression of recombinant immunoglobulins which may be utilized in carrying out the present invention: Weidle et al., Gene, 51: 21-29 (1987); Dorai et al., J. Immunol., 13(12):4232-4241 (1987); De Waele et al., Eur. J. Biochem., 176:287-295 (1988); Colcher et al., Cancer Res., 49:1738-1745 (1989); Wood et al., J. Immunol., 145(a):3011-3016 (1990); Bulens et al., Eur. J. Biochem., 195:235-242 (1991); Beggington et al., Biol. Technology, 10:169 (1992); King et al., Biochem. J., 281:317-323 (1992); Page et al., Biol. Technology, 2:64 (1991); King et al., Biochem. J., 290:723-729 (1993); Chaudary et al., Nature, 339:394-397 (1989); Jones et al., Nature, 321:522-525 (1986); Morrison and Oi, Adv. Immunol., 44:65-92 (1988); Benhar et al., Proc. Natl. Acad. Sci. USA, 91:12051-12055 (1994); Singer et al., J. Immunol., 150:2844-2857 (1993); Cooto et al., Hybridoma, 13(3):215-219 (1994); Queen et al., Proc. Natl. Acad. Sci. USA, 86:10029-10033 (1989); Caron et al., Cancer Res., 32:6761-6767 (1992); Cotoma et al., J. Immunol. Meth., 152:89-109 (1992). Moreover, vectors suitable for expression of recombinant antibodies are commercially available.

Host cells capable of expressing functional immunoglobulins include, e.g., mammalian cells such as Chinese Hamster Ovary (CHO) cells; COS cells; myeloma cells, such as NSO and SP2/O cells; bacteria such as Escherichia coli; yeast cells such as Saccharomyces cerevisiae; and other host cells.

3. Single Chain Antibodies

In some embodiments, the antibodies of the invention are single chain Fvs (scFvs). The V_{H} and the V_{L} regions (e.g., SEQ ID NO:1 and SEQ ID NO:2, or SEQ ID NO:3 and SEQ ID NO:4) of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length.

Methods of making scFv antibodies have been described. See, e.g., Huse et al., Science 246:1275-1281 (1989); Ward et al., Nature 341:544-546 (1989); and Vaughan et al., Nature Biotech. 14:309-314 (1996). In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to TrkB coated plates or to cells expressing TrkB on their surface are expanded in E. coli and subjected to another round of panning. In this way, an enrichment of many fold occurs in 3 rounds of panning. Thus, even when enrichment in each round is low, multiple rounds of panning will lead to the isolation of rare phage and the genetic material contained within which encodes the scFv with the highest affinity or one which is better expressed on phage.

Regardless of the method of panning chosen, the physical link between genotype and phenotype provided by phage display makes it possible to test every member of a cDNA library for binding to antigen, even with large libraries of clones.

4. Human Antibodies

In some embodiments, human antibodies are used according to the present invention. Human antibodies can be made by a variety of methods known in the art including by using phage display methods using antibody libraries derived from human immunoglobulin sequences. See, e.g., Lonberg and Huszar, Int. Rev. Immunol. 13:65-93 (1995), U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

In some embodiments, the antibodies of the present invention are generated using phage display. For example, functional antibody domains are displayed on the surface of phage particles that carry the polynucleotide sequences encoding them. Such phage can be utilized to display antigen-binding domains expressed from a repertoire or combinatorial antibody library (e.g., human or murine). Phage expressing an antigen binding domain that binds TrkB can be selected or identified with TrkB, e.g., using labeled TrkB. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

5. Generating Agonist Antibodies

Agonist antibodies can be identified by generating anti-TrkB antibodies and then testing each antibody for the ability trigger TrkB mediated events, e.g., initiating differentiation and/or dendrification of SH-SY5Y cells, measuring Anoikis (apoptosis resulting from loss of cell-matrix interactions) in response to treatment of cells with potential TrkB agonist, or using a BaF3/TrkB cell proliferation assay.

SH-SY5Y assays involve plating out SH-SY5Y cells and treating the cells with retenoic acid with or without potential agonist antibodies and/or BDNF and subsequently measure neurite outgrowth. Generally, retinoic acid alone will induce a small amount of neurite outgrowth. BDNF alone should not induce significant neurite outgrowth and antibody alone should not induce significant neurite outgrowth. However, cells treated with retinoic acid, BDNF, and antibody should exhibit extensive neurite outgrowth. An exemplary SH-SY5Y assay is described in Kaplan D R, et al., Neuron 11:321-331 (1993).

BaF3/TrkB cell proliferation assays involve measuring proliferation of cells stimulates by agonism of the TrkB receptor. For example, BaF3 cells are grown in complete RPMI medium with 1 IL-3 and infected with TrkB retrovirus. Cells are washed in the absences of IL-3 and plated. Potential agonist antibodies and cell survival is measured (e.g., using luminescent cell viability detection reagent such as Cell-Titer Glo™) after an appropriate incubation. Positive control cells are incubated with rhBDNF.

Anoikis assays involve resuspending RIE/TrkB cells (e.g., in DMEM medium) and contacting cells, optionally in multi-well containers, with a potential antibody agonist (e.g., 2.5×10⁴ cells 10 μl of 1-20 μg/ml antibody). The mixtures are incubated in the presence or absence of a hBDNF control and then measured for cell viability (e.g., using luminescent cell viability detection reagent such as Cell-Titer Glo™). An exemplary Anoikis assay is described in Douma et al., Nature 430:1034-1039 (2004).

TrkB agonists can also be evaluated on SH-SY5Y cells for their ability to protect cells from vinblastine and cisplatin toxicity. This assays has been described in, e.g., Scala et al., Cancer Res. 56(16):3737-42 (1996); and Jaboin et al., Cancer Res. 62(22):6756-63 (2002).

### III. Antibody Uses

The TrkB agonists antibodies of the invention can be used to treat or ameliorate any diseases or conditions that benefit from increased TrkB activity.

In some embodiments, the TrkB agonist antibodies of the invention are used to treat or alleviate hyperglycemia and/or diabetes or symptoms thereof in an individual. Alternatively, or in combination, the antibodies of the invention can be used to reduce weight in an individual in need thereof. In some embodiments, the antibodies are used to alleviate obesity. This can be particularly useful as obese individuals are more prone to insulin resistance and type II diabetes.

The present invention also provides for methods of treatment or prevention of neurodegenerative or central nervous system (CNS) diseases by administration of the TrkB agonist antibodies of the invention to an individual in need thereof. Exemplary CNS diseases include, e.g., Alzheimer's, Parkinson's, Huntington's or ALS diseases.

Increasing TrkB activation has also be implicated in alleviation of substance abuse. See, e.g., U.S. Patent Publication No. 2005/0203011. Accordingly, the present invention provides for methods of alleviating substance (e.g., alcohol, nicotine and/or narcotics) abuse and dependence by administering the TrkB agonist antibodies of the invention to an individual in need there of.

The antibodies and agents of the invention can be administered directly to the mammalian subject in need thereof. Administration of the compositions of the present invention is by any of the routes normally used for introducing pharmaceuticals, including antibodies, into ultimate contact with the tissue to be treated. The antibodies are administered in any suitable manner, optionally with pharmaceutically acceptable carriers. Suitable methods of administering such antibodies and agents are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. 1985)).

The antibodies, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, by orally, topically, intravenously, intraperitoneally, intravesically or intrathecally. Optionally, the compositions are administered nasally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug. The compounds of the present invention can also be used effectively in combination with one or more additional active agents (e.g., chemotherapeutics) depending on the desired therapy or effect.

The dose administered to a patient, in the context of the present invention should be sufficient to effect a beneficial response in the subject over time. The dose will be determined by the efficacy of the particular antibodies and reagents employed and the condition of the subject, as well as the body weight or surface area of the area to be treated. The size of the dose also will be determined by the existence, nature, and extent of any adverse side-effects that accompany the administration of a particular compound or vector in a particular subject. Administration can be accomplished via single or divided doses.

The TrkB antibody agonist can be used in combination with agents known to be beneficial for reducing weight, reducing blood glucose levels, treat diabetes or alleviate diabetic symptoms, treating neurodegenerative diseases or reducing substance abuse. Exemplary agents used to treat diabetes include, e.g., insulin; sulfonylureas (e.g. Glipizide and Amaryl) and insulinotropic agents (e.g. nateglinide and repaglinide); metformin; PPARgamma agonists (e.g. rosiglitizone and pioglitazone) as well as PPARalpha, PPARdelta, PPARalpha/gamma dual agonists and PPARalpha/gamma/delta pan agonists; alpha-glucosidase inhibitors (e.g. Acarbose); DPP-IV inhibitors (e.g. vildagliptin); and GLP-1/GLP-1 analogs (e.g. exenatide). Exemplary agents used to treat obesity include, e.g., lipase inhibitors (e.g. orlistat); sibutramine; CB-1 inhibitors (e.g. rimonabant); topiramate; amylin/amylin analogs (e.g. pramlintide), leptin, PYY/PYY analogs; and GLP-1/GLP-1 analogs (e.g. exenatide).

Active agents that can be administered together in a mixture with the TrkB agonist antibody or each can be administered separately. The antibody agent and the other active agent can, but need not, be administered concurrently.

### EXAMPLE

### Example 1

This example discusses the identification and characterization of antibody agonists of TrkB.

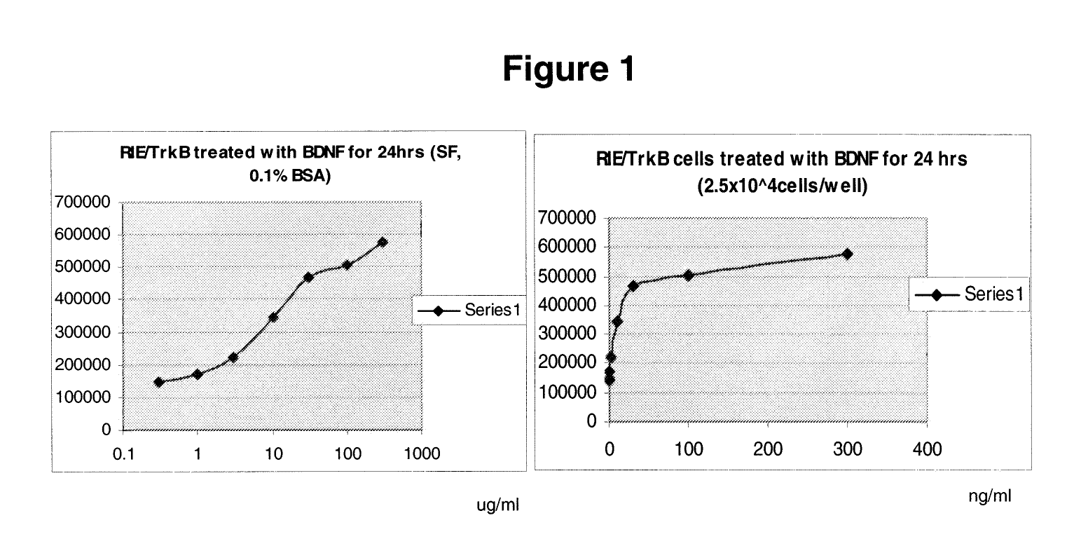

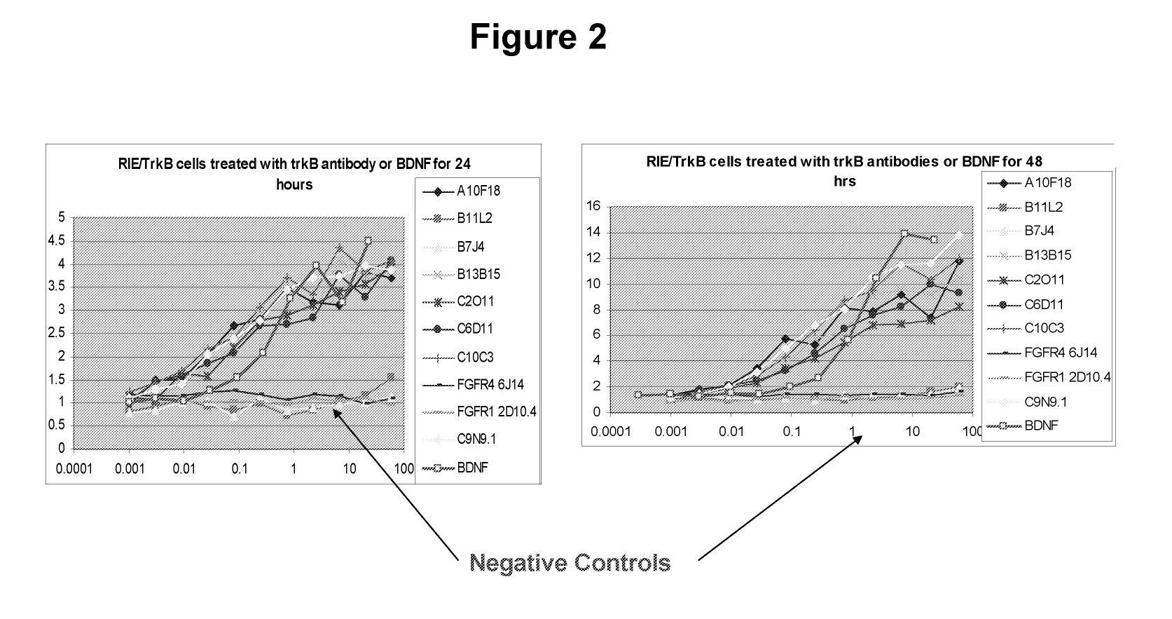

Mice were immunized with human trkB receptor and hybridoma supernatants from serum IgG positive mice were screened for reactivity to TrkB by ELISA. Resulting antibodies were screened for their ability to activate TrkB by testing the antibodies' ability to rescue RIE/TrkB cells from Anoikas. BDNF is known to rescue RIE/TrkB cells from Anoikas and therefore this assay is a good measure of an antibody's ability to activate TrkB. See, FIG. 1. Multiple TrkB antibody agonists were identified that mimic the activity of BDNF in an Anoikis assay, as shown in FIG. 2. Positive antibody agonists were purified from low IgG media.

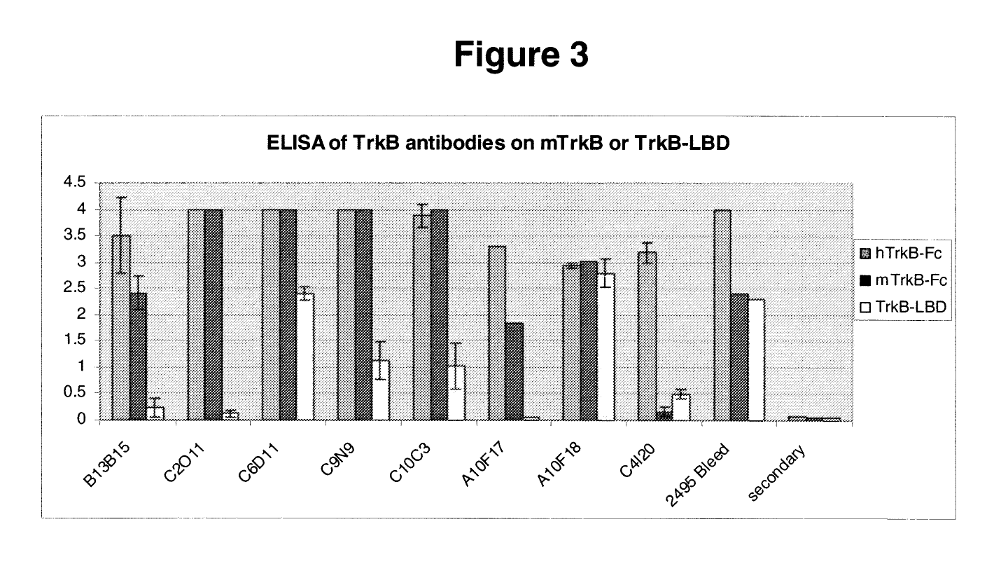

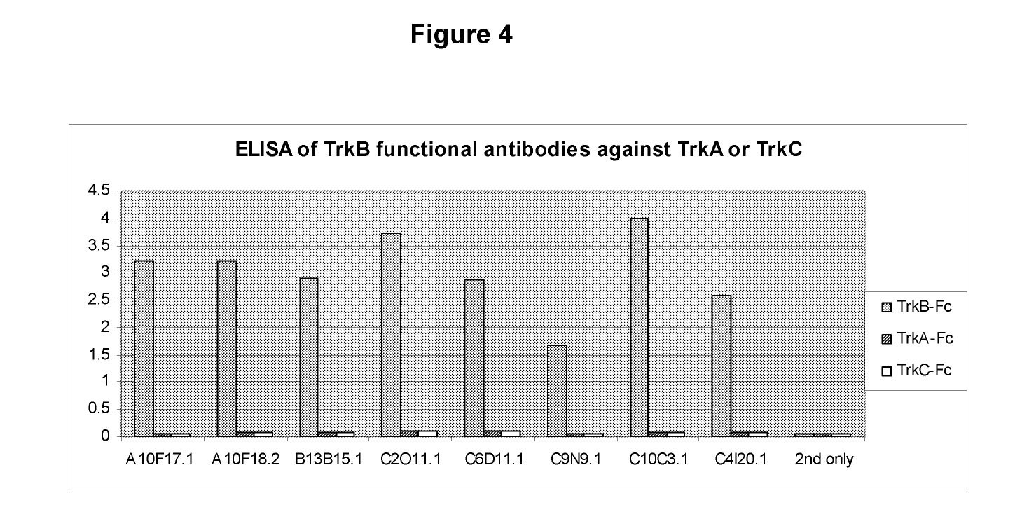

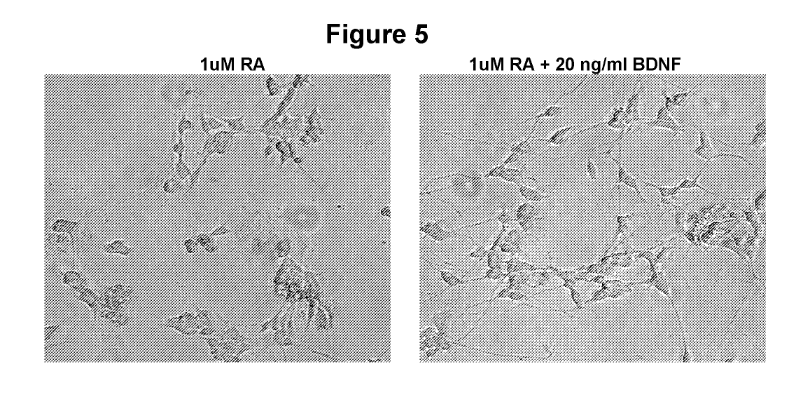

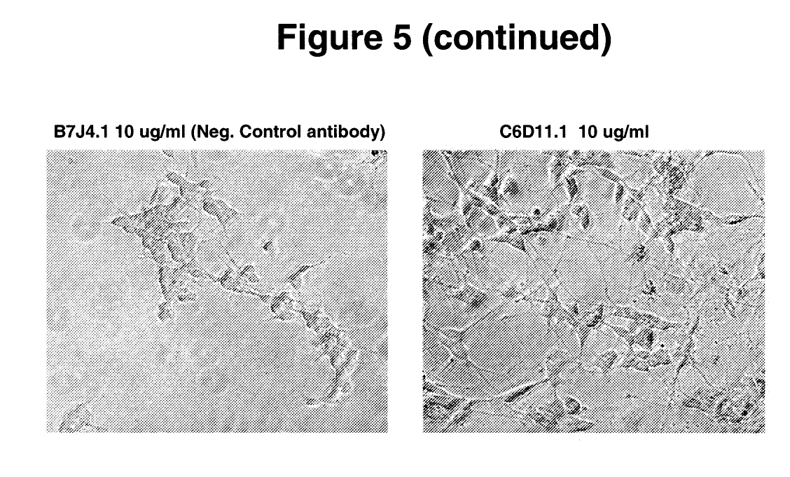

Antibody agonists were screened for reactivity to huTrkB, muTrkB, and TrkB ligand binding domain (LBD), as shown in FIG. 3. Most of the agonists did not bind to the LBD. In particular, it was found that C20 is reactive with mouse and human trkB. A10 is reactive with mouse and human trkB and binds to the ligand binding domain epitope. As shown in FIG. 4, many of the functional antibodies were shown to be reactive against mouse Trk B, but did not bind to TrkA, or TrkC.

Antibody agonists were confirmed in a neurite outgrowth in vitro model. The results showed that TrkB antibody agonists stimulate neurite outgrowth similar to BDNF.

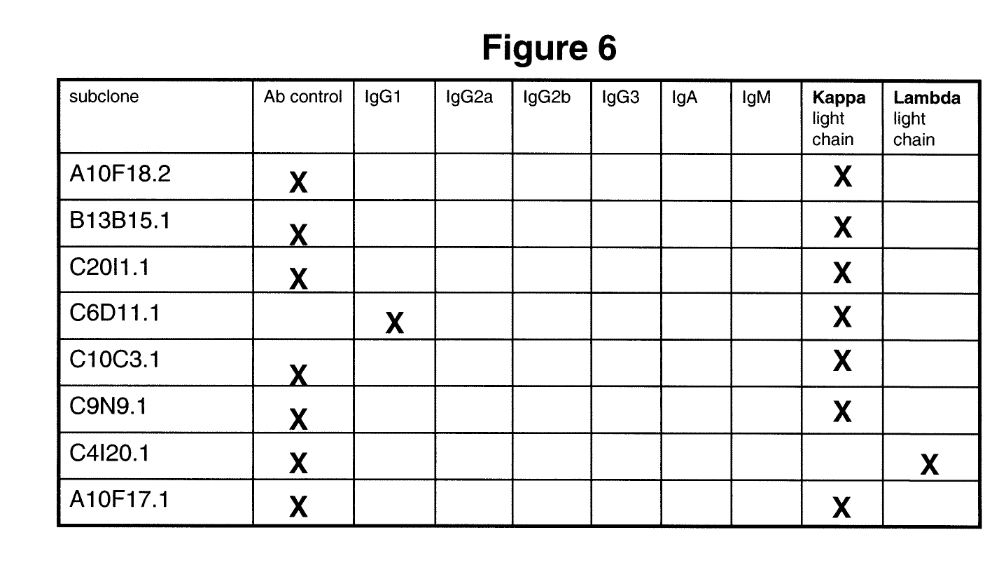

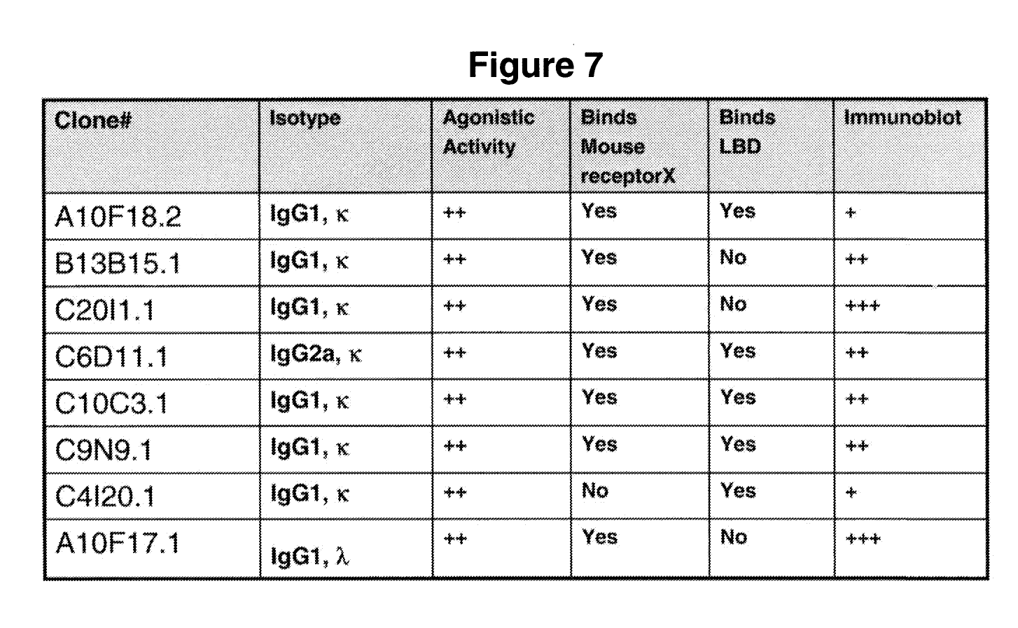

The functional antibodies were isotyped as shown in FIG. 6. The functional antibodies were further shown to bind to human TrkB over-expressed in RIE cells by western blots. FIG. 7 summarizes information for various agonist antibodies identified.

### Example 2

This example shows that TrkB agonist antibodies effectively reduce blood glucose and weight in mice.

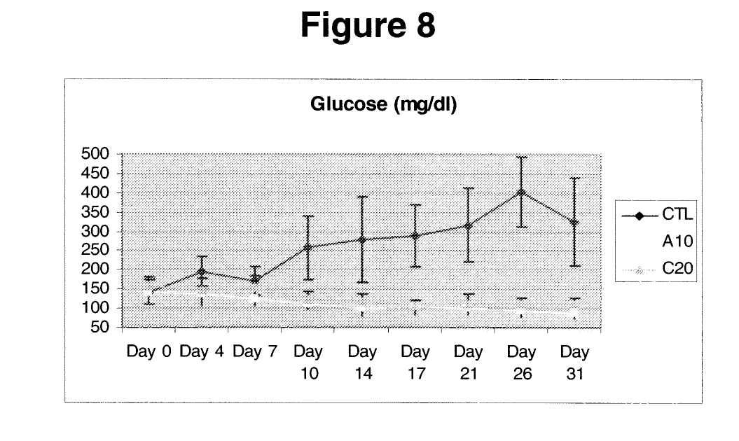

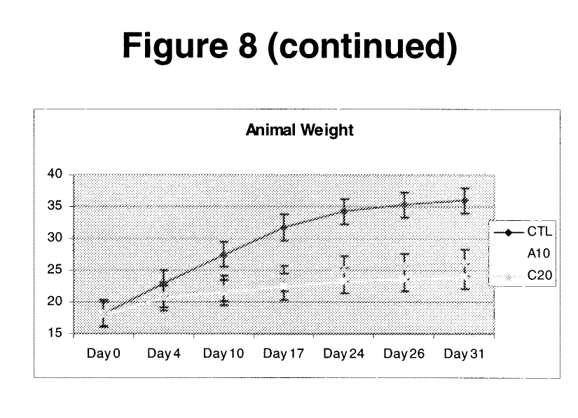

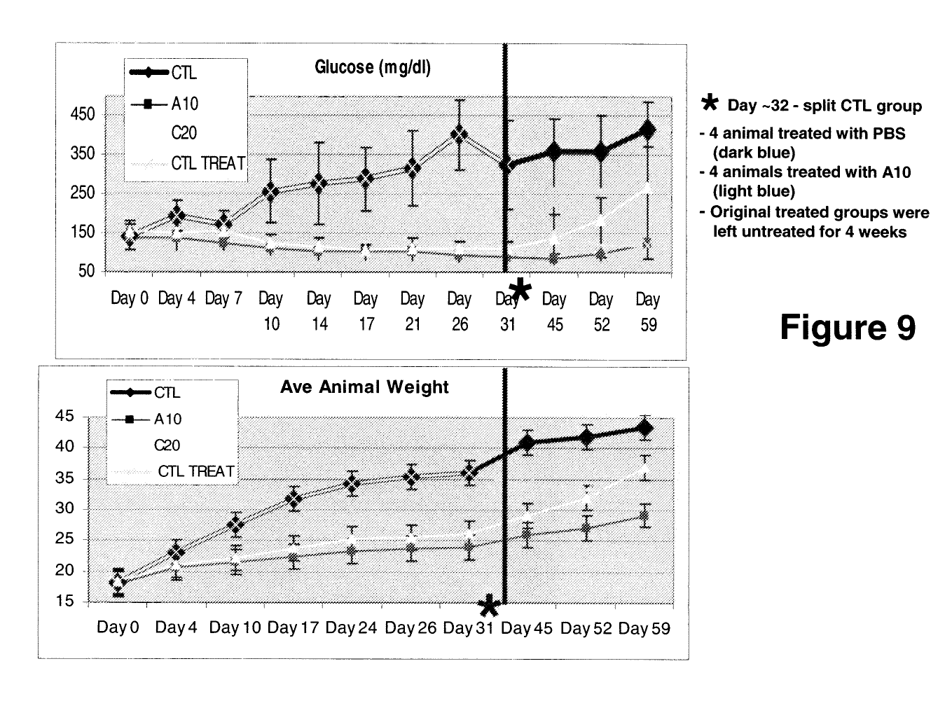

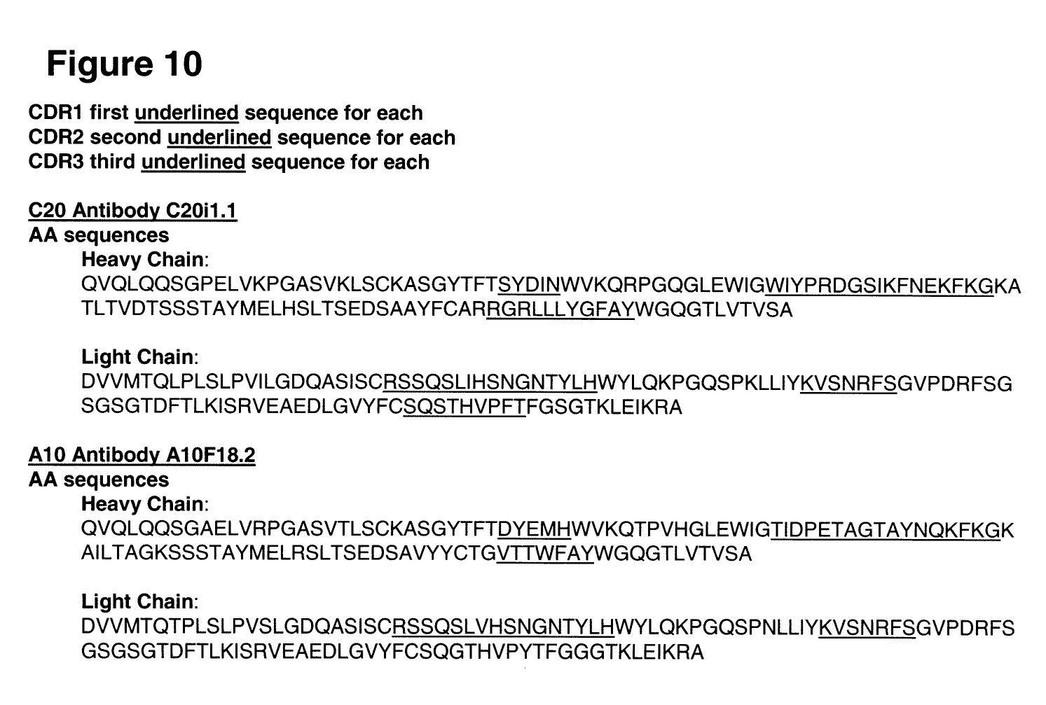

The two most potent agonists, A10 and C20 (whose variable regions are displayed in SEQ ID NOs:3 and 4 and SEQ ID NOs: 1 and 2, respectively), were tested in a db/db mouse model of type 2 diabetes. As shown in FIGS. 8 and 9, both antibodies appeared to reduce serum glucose levels and promote weight loss prophylactically and therapeutically.

As shown in the top graph of FIG. 9, mice prone to high blood glucose levels did not have increased blood glucose levels when antibodies A10 or C20 were administered, whereas control mice did have elevated levels. This result shows that the antibodies have a prophylactic effect. Thirty-two days into the study the hyperglycemic, obese control animals (8) were split into 2 groups. One group was treated with antibody A10 and the other was treated with PBS. Treatment reversed hyperglycemia within 1 week and reduced weight by 25%. The original treated animals were left untreated for 4 additional weeks. The C20 treated group had increased glucose levels and increase weight gain. The A10 group maintained normal glucose and showed a small weight increase. These results are shown in the top and bottom panels of FIG. 9. A PK study revealed that the A10 antibody had a much better serum half-life, which corresponds to the effects seen in vivo.

Although the invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, Genbank sequences, patents, and patent applications cited in this specification are herein incorporated by reference as if each was specifically and individually indicated to be incorporated by reference.

What is claimed is:

1. An isolated antibody agonist of Tyrosine Kinase Receptor B (TrkB).

2. The antibody of claim 1, wherein the antibody is a humanized antibody.

3. The antibody of claim 1, wherein the antibody is a single chain antibody.

4. The antibody of claim 1, wherein the antibody does not bind to Tyrosine Kinase Receptor A or Tyrosine Kinase Receptor C.

5. The antibody of claim 1, wherein the antibody binds to the Ligand Binding Domain (LBD) of TrkB.

6. The antibody of claim 1, wherein the antibody competes with the binding of Brain Derived Neurotrophic Factor (BDNF) to TrkB.

7. The antibody of claim 1, wherein the antibody comprises
i. a heavy chain variable region comprising SEQ ID NO:7; and
ii. a light chain variable region comprising SEQ ID NO:8.

8. The antibody of claim 7, wherein the antibody comprises
i. a heavy chain variable region comprising SEQ ID NO:7, SEQ ID NO:11, and SEQ ID NO:15; and
ii. a light chain variable region comprising SEQ ID NO:8, SEQ ID NO:12, and SEQ ID NO:16.

9. The antibody of claim 8, wherein the antibody comprises
i. a heavy chain variable region comprising SEQ ID NO:3; and
ii. a light chain variable region comprising SEQ ID NO:4.

10. The antibody of claim 1, wherein the antibody does not bind to the LBD of TrkB.

11. The antibody of claim 1, wherein the antibody does not compete with the binding of BDNF to TrkB.

12. The antibody of claim 1, wherein the antibody comprises
i. a heavy chain variable region comprising SEQ ID NO:5; and
ii. a light chain variable region comprising SEQ ID NO:6.

13. The antibody of claim 12, wherein the antibody comprises
i. a heavy chain variable region comprising SEQ ID NO:5, SEQ ID NO:9, and SEQ ID NO:13; and
ii. a light chain variable region comprising SEQ ID NO:6, SEQ ID NO:10, and SEQ ID NO:14.

14. The antibody of claim 13, wherein the antibody comprises
i. a heavy chain variable region comprising SEQ ID NO:1; and
ii. a light chain variable region comprising SEQ ID NO:2.

15. A pharmaceutical composition comprising
i. a therapeutically effective amount of the antibody of claim 1; and
ii. a pharmaceutical carrier.

16. The pharmaceutical composition of claim 15, wherein the antibody is selected from the group consisting of:
i. an antibody comprising a heavy chain variable region comprising SEQ ID NO:5 and a light chain variable region comprising SEQ ID NO:6; and
ii. an antibody comprising a heavy chain variable region comprising SEQ ID NO:7 and a light chain variable region comprising SEQ ID NO:8.

17. The pharmaceutical composition of claim 15, wherein the pharmaceutical composition further comprises an agent that reduces blood glucose levels and/or body weight in an individual.

18. A method of reducing blood glucose levels and/or body weight in an individual in need thereof, the method comprising administering a therapeutically effective amount of the antibody of claim 1 to the individual.

19. The method of claim 18, wherein the individual has a condition selected from the group consisting of: pre-diabetes, type I diabetes, type II diabetes, being overweight, and obesity.

20. The method of claim 18, wherein a therapeutically effective amount of a second agent effective in reducing blood glucose and/or body weight is administered to the individual in combination with the antibody of claim 1.

21. The method of claim 20, wherein the second agent and the antibody of claim 1 are administered as a mixture.

22. The method of claim 20, wherein the second agent is administered separately from the antibody of claim 1.

23. The method of claim 20, wherein the second agent is selected from the group consisting of: insulin, sulfonylureas, insulinotropic agents, metformin, PPARγ agonists PPARα agonists, PPARδ agonists, PPARα/δ dual agonists, PPARα/γ/δ pan agonists, alpha-glucosidase inhibitors, DPP-IV inhibitors, lipase inhibitors, sibutramine, CB-1 inhibitors, topiramate, amylin, amylin analogs, leptin, PYY/PYY analogs, and GLP-1/GLP-1 analogs.

24. The method of claim 18, wherein the antibody is a humanized antibody.

25. The method of claim 18, wherein the antibody is selected from the group consisting of:
i. an antibody comprising the heavy chain variable region comprising SEQ ID NO:5 and a light chain variable region comprising SEQ ID NO:6; and
ii. an antibody comprising a heavy chain variable region comprising SEQ ID NO:7 and a light chain variable region comprising SEQ ID NO:8.


SEQUENCE LISTING (18 sequences)

SEQ ID NO: 1 - protein, 120 aa, Artificial Sequence
  Description of Artificial Sequence Synthetic polypeptide
QVQLQQSGPELVKPGASVKLSCKASGYTFTSYDINWVKQRPGQGLEWIGWIYPRDGSIKFNEKFKGKATLTVDTSSSTAYMELHSLTSEDSAAYFCARRGRLLLYGFAYWGQGTLVTVSA

SEQ ID NO: 2 - protein, 114 aa, Artificial Sequence
  Description of Artificial Sequence Synthetic polypeptide
DVVMTQLPLSLPVILGDQASISCRSSQSLIHSNGNTYLHWYLQKPGQSPKLLIYKVSNRFSGVPDRFSGSGSGTDFTLKISRVEAEDLGVYFCSQSTHVPFTFGSGTKLEIKRA

SEQ ID NO: 3 - protein, 116 aa, Artificial Sequence
  Description of Artificial Sequence Synthetic polypeptide
QVQLQQSGAELVRPGASVTLSCKASGYTFTDYEMHWVKQTPVHGLEWIGTIDPETAGTAYNQKFKGKAILTAGKSSSTAYMELRSLTSEDSAVYYCTGVTTWFAYWGQGTLVTVSA

SEQ ID NO: 4 - protein, 114 aa, Artificial Sequence
  Description of Artificial Sequence Synthetic polypeptide
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQGTHVPYTFGGGTKLEIKRA

SEQ ID NO: 5 - protein, 11 aa, Artificial Sequence
  Description of Artificial Sequence Synthetic peptide
RGRLLLYGFAY

SEQ ID NO: 6 - protein, 9 aa, Artificial Sequence
  Description of Artificial Sequence Synthetic peptide
SQSTHVPFT

SEQ ID NO: 7 - protein, 7 aa, Artificial Sequence
  Description of Artificial Sequence Synthetic peptide
VTTWFAY

SEQ ID NO: 8 - protein, 9 aa, Artificial Sequence
  Description of Artificial Sequence Synthetic peptide
SQGTHVPYT

SEQ ID NO: 9 - protein, 5 aa, Artificial Sequence
  Description of Artificial Sequence Synthetic peptide
SYDIN

SEQ ID NO: 10 - protein, 16 aa, Artificial Sequence
  Description of Artificial Sequence Synthetic peptide
RSSQSLIHSNGNTYLH

SEQ ID NO: 11 - protein, 5 aa, Artificial Sequence
  Description of Artificial Sequence Synthetic peptide
DYEMH

SEQ ID NO: 12 - protein, 16 aa, Artificial Sequence
  Description of Artificial Sequence Synthetic peptide
RSSQSLVHSNGNTYLH

SEQ ID NO: 13 - protein, 17 aa, Artificial Sequence
  Description of Artificial Sequence Synthetic peptide
WIYPRDGSIKFNEKFKG

SEQ ID NO: 14 - protein, 7 aa, Artificial Sequence
  Description of Artificial Sequence Synthetic peptide
KVSNRFS

SEQ ID NO: 15 - protein, 17 aa, Artificial Sequence
  Description of Artificial Sequence Synthetic peptide
TIDPETAGTAYNQKFKG

SEQ ID NO: 16 - protein, 7 aa, Artificial Sequence
  Description of Artificial Sequence Synthetic peptide
KVSNRFS

SEQ ID NO: 17 - protein, 477 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGFVLFHKIPLDG

SEQ ID NO: 18 - protein, 144 aa, Homo sapiens
PTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREH